(19) United States
(12) Patent Application Publication
(10) Pub. No.: US 2010/0047862 A1
(43) Pub. Date: Feb. 25, 2010

(54) Novel DNA Polymerase

(76) Inventors: Yoshihide Hayashizaki, Saitama (JP); Masayoshi Itoh, Saitama (JP); Yoshimi Benno, Ibaraki (JP); Alexander Lezhava, Kanagawa (JP)

Correspondence Address: BIRCH STEWART KOLASCH & BIRCH, PO BOX 747, FALLS CHURCH, VA, 22040-0747, US

(21) Appl. No.: 12/083,695

(22) PCT Filed: Oct. 20, 2006
(86) PCT No.: PCT/JP2006/321455

§ 371 Date: Apr. 17, 2008

(30) Foreign Application Priority Data
Oct. 20, 2005 (JP) 2005-306228

(51) Int. Cl. C12P 21/00; C12N 9/12; C07H 21/00; C12N 15/63; C12N 1/00; C12P 19/34

(52) U.S. Cl. 435/69.1; 435/194; 536/23.2; 435/320.1; 435/243; 435/91.2; 536/24.33

(57) ABSTRACT

This invention provides a novel DNA polymerase obtained from Bacillus smithii JCM9076, which has novel features in terms of, for example, optimal reaction conditions (e.g., optimal temperature) and enzyme activity. More particularly, a novel DNA polymerase is a pol I type DNA polymerase, which is any of proteins (a) to (f) below and has DNA polymerase activity: (a) a protein comprising the amino acid sequence as shown in SEQ ID NO: 7; (b) a protein consisting of an amino acid sequence derived from the amino acid sequence as shown in SEQ ID NO: 7 by deletion, substitution, or addition of one or several amino acid residues; (c) a protein consisting of the amino acid sequence as shown in SEQ ID NO: 9; and (d) a protein consisting of an amino acid sequence derived from the amino acid sequence as shown in SEQ ID NO: 9 by deletion, substitution, or addition of one or several amino acid residues.

### TECHNICAL FIELD

The present invention relates to novel DNA polymerase derived from Bacillus smithii, which has strand-displacement activity.

### BACKGROUND ART

DNA polymerase is an enzyme that has been most commonly used in the life science field. DNA polymerase is an enzyme that is essential for a variety of techniques, including PCR. A wide variety of DNA polymerases are sold, and each such polymerase has its own reaction condition and enzyme activity characteristics. PCR is generally employed in order to amplify DNA; however, PCR suffers from problems, in that it requires the use of a thermal cycler for complicated temperature control and it requires several hours. As alternative techniques for DNA amplification, the LAMP method, the SDA method, and other methods have been developed, although DNA polymerase having strand-displacement activity is required for such reactions. DNA polymerases having strand-displacement activity have been reported (see Japanese Patent No. 2,978,001); however, the variety of such DNA polymerases that are commercially available at present is small. Because of limiting reaction conditions, such as optimal temperature or long reaction duration, development of test or diagnostic agents involving the use of such DNA amplification techniques has also been restricted.

### DISCLOSURE OF THE PRESENT INVENTION

The present invention provides novel DNA polymerase, which is obtained from the Bacillus smithii JCM9076 strain and has novel features such as optimal reaction conditions (e.g., optimal temperature) and enzyme activity.

DNA polymerase is an enzyme that has been most commonly used in the life science field. DNA polymerase is an enzyme that has been most commonly used in the life science field. DNA polymerase is an enzyme that is essential for a variety of techniques, including PCR. A wide variety of DNA polymerases are sold, and each such polymerase has its own reaction condition and enzyme activity characteristics. PCR is generally employed in order to amplify DNA; however, PCR suffers from problems, in that it requires the use of a thermal cycler for complicated temperature control and it requires several hours. As alternative techniques for DNA amplification, the LAMP method, the SDA method, and other methods have been developed, although DNA polymerase having strand-displacement activity is required for such reactions. DNA polymerases having strand-displacement activity have been reported (see Japanese Patent No. 2,978,001); however, the variety of such DNA polymerases that are commercially available at present is small. Because of limiting reaction conditions, such as optimal temperature or long reaction duration, development of test or diagnostic agents involving the use of such DNA amplification techniques has also been restricted.

The present inventors isolated a novel pol I type DNA polymerase from Bacillus smithii, which has common activity of an enzyme for template-dependent DNA replication, activity of an enzyme for complementary strand-displacement replication, and reverse transcriptase activity. The present inventors discovered that such DNA polymerase has properties superior to those of conventional DNA polymerases. This has led to the completion of the present invention.

Specifically, the present invention is as follows.

[1] Pol I type DNA polymerase, which is any of proteins (a) to (f) below and has DNA polymerase activity:

(a) a protein comprising the amino acid sequence as shown in SEQ ID NO: 7;

(b) a protein consisting of an amino acid sequence derived from the amino acid sequence as shown in SEQ ID NO: 7 by deletion, substitution, or addition of one or several amino acid residues;

(c) a protein consisting of the amino acid sequence as shown in SEQ ID NO: 9;

(d) a protein consisting of an amino acid sequence derived from the amino acid sequence as shown in SEQ ID NO: 9 by deletion, substitution, or addition of one or several amino acid residues;

(e) a protein consisting of an amino acid sequence derived from the amino acid sequence as shown in SEQ ID NO: 7 by deletion of a consecutive amino acid sequence from 1st Val to any amino acid up to 297th Glu; and

(f) a protein consisting of an amino acid sequence derived by deletion, substitution, or addition of one or several amino acid residues from the amino acid sequence derived from the amino acid sequence as shown in SEQ ID NO: 7 by deletion of a consecutive amino acid sequence from 1st Val to any amino acid up to 297th Glu.

[2] DNA encoding any of proteins (a) to (f) below having DNA polymerase activity:

(a) a protein comprising the amino acid sequence as shown in SEQ ID NO: 7;

(b) a protein consisting of an amino acid sequence derived from the amino acid sequence as shown in SEQ ID NO: 7 by deletion, substitution, or addition of one or several amino acid residues;

(c) a protein consisting of the amino acid sequence as shown in SEQ ID NO: 9;

(d) a protein consisting of an amino acid sequence derived from the amino acid sequence as shown in SEQ ID NO: 9 by deletion, substitution, or addition of one or several amino acid residues;

(e) a protein consisting of an amino acid sequence derived from the amino acid sequence as shown in SEQ ID NO: 7 by deletion of a consecutive amino acid sequence from 1st Val to any amino acid up to 297th Glu; and

(f) a protein consisting of an amino acid sequence derived by deletion, substitution, or addition of one or several amino acid residues from the amino acid sequence derived from the amino acid sequence as shown in SEQ ID NO: 7 by deletion of a consecutive amino acid sequence from 1st Val to any amino acid up to 297th Glu.

[3] DNA, which is any of (g) to (j) below encoding a protein having DNA polymerase activity:

(g) DNA consisting of the nucleotide sequence as shown in SEQ ID NO: 6;

(h) DNA hybridizing under stringent conditions to DNA consisting of a sequence complementary to DNA consisting of the nucleotide sequence as shown in SEQ ID NO: 6 and encoding a protein;

(i) DNA consisting of the nucleotide sequence as shown in SEQ ID NO: 8; and

(j) DNA hybridizing under stringent conditions to DNA consisting of a sequence complementary to DNA consisting of the nucleotide sequence as shown in SEQ ID NO: 8 and encoding a protein.

[4] A recombinant vector comprising DNA according to [2] or [3].

[5] A transformant comprising the recombinant vector according to [4].

[6] A method for producing a pol I type DNA polymerase which comprises culturing the transformant according to [5] and sampling the pol I type DNA polymerase from the culture product.

[7] The pol I type DNA polymerase according to [1] having activity of an enzyme for complementary strand-displacement replication and reverse transcriptase activity.

[8] The pol I type DNA polymerase according to [1] or [7] lacking 5′→3′ exonuclease activity.

[9] The pol I type DNA polymerase according to any of [1], [7], or [8] having 3′→5′ exonuclease activity.

[10] The pol I type DNA polymerase according to any of [1], [7], or [8] lacking 3′→5′ exonuclease activity.

[11] A method for nucleic acid amplification using the pol I type DNA polymerase according to any of [1] and [7] to [10].

[12] The method for nucleic acid amplification according to [11], which is an isothermal amplification method.

[13] A kit for nucleic acid amplification comprising the pol I type DNA polymerase according to any of [1] and [7] to [10].

[14] A method for cloning the pol I type DNA polymerase gene comprising steps of:

(1) preparing a primer consisting of the nucleotide sequence as shown in SEQ ID NO: 1 and a primer consisting of the nucleotide sequence as shown in SEQ ID NO: 2;

(2) preparing a genomic DNA template;

(3) amplifying a genomic DNA template using a primer consisting of the nucleotide sequence as shown in SEQ ID NO: 1 and a primer consisting of the nucleotide sequence as shown in SEQ ID NO: 2;

(4) cloning the amplified fragment of (3);

(5) amplifying DNA encoding pol I type DNA polymerase using a primer consisting of the nucleotide sequence as shown in SEQ ID NO: 3 and a primer consisting of the nucleotide sequence as shown in SEQ ID NO: 4; and

(6) cloning the amplified fragment of (5).

[15] A method for cloning the pol I type DNA polymerase gene consisting of the sequence as shown in SEQ ID NO: 8 which comprises steps of:

(1) preparing a primer consisting of the nucleotide sequence as shown in SEQ ID NO: 4 and a primer consisting of the nucleotide sequence as shown in SEQ ID NO: 5;

(2) preparing a genomic DNA template;

(3) amplifying a genomic DNA template using a primer consisting of the nucleotide sequence as shown in SEQ ID NO: 4 and a primer consisting of the nucleotide sequence as shown in SEQ ID NO: 5; and

(4) cloning the amplified fragment.

[16] A primer consisting of a fragment of the DNA according to [2] or [3] and comprising 5 to 50 nucleotides.

This description includes part or all of the contents as disclosed in the description and/or drawings of Japanese Patent Application No. 2005-306228, which is a priority document of the present application.

### BRIEF DESCRIPTION OF THE DRAWINGS

FIG. 1 shows the results of DNA polymerase activity assay showing the regression line representing the amount of DNA polymerase and the assayed value.

FIG. 2 shows the results of assaying activity of an enzyme for complementary strand-displacement replication using the enzyme of the present invention (i.e., Bsm DNA polymerase).

FIG. 3 shows the results of assaying reverse transcriptase activity using the enzyme of the present invention (i.e., Bsm DNA polymerase).

FIG. 4 shows the results of assaying reverse transcriptase activity (with the use of a ladder) using the enzyme of the present invention (i.e., Bsm DNA polymerase).

FIG. 5 shows the results of isothermal gene amplification using the enzyme of the present invention (i.e., Bsm DNA polymerase).

FIG. 6 shows the positional relationship between a template sequence and a primer at the time of isothermal gene amplification.

### BEST MODES FOR CARRYING OUT THE INVENTION

The DNA polymerase of the present invention can be isolated from a Bacillus microorganism, preferably from Bacillus smithii, and more preferably from a Bacillus smithii JCM9076 strain. The Bacillus smithii JCM9076 strain can be obtained from the Japan Collection of Microorganisms (JCM), The RIKEN BioResource Center (RIKEN BRC) (http://www.jcm.riken.jp/JCM/JCM_Home_J.html).

In the present invention, DNA can be obtained in accordance with a method described in publications well-known in the art, such as J. Sambrook, E. F. Fritsch & T. Maniatis, 1989, Molecular Cloning, a laboratory manual, second edition, Cold Spring Harbor Laboratory Press; and Ed Harlow and David Lanc, 1988, Antibodies, a laboratory manual, Cold Spring Harbor Laboratory Press.

DNA that encodes the pol I type DNA polymerase of the present invention can be isolated from a Bacillus microorganism by comparing DNA sequences of known pol I type DNA polymerases, synthesizing primers based on the nucleotide sequences of conserved regions having common sequences, and using the resulting primers. Also, phoR and mutM are conserved in regions upstream and downstream of the gene that encodes the pol I type DNA polymerase of a Bacillus microorganism. Accordingly, primers may be designed based on the sequences of such conserved regions. Genes are amplified by PCR using such primers. Subsequently, the amplified product is cloned, the sequence is determined, and a pair of primers designed to sandwich the ORF region may be used to further amplify the gene. When a region equivalent to the Klenow fragment of the E. coli DNA polymerase I of the pol I type DNA polymerase is to be isolated, one of the primers is designed based on the nucleotide sequence of the region equivalent to the N-terminus of the E. coli Klenow fragment, and the pair of primers may be used so as to sandwich the region equivalent to the Klenow fragment.

The present invention includes a method for isolating a pol I type DNA polymerase from a microorganism using such primers. An example of a pair of primers that can be used is a pair of a primer consisting of the nucleotide sequence as shown in SEQ ID NO: 1 and a primer consisting of the nucleotide sequence as shown in SEQ ID NO: 2 that were designed based on the sequences of the conserved region. As primers that sandwich the ORF region of the pol I type DNA polymerase, a pair of a primer consisting of the nucleotide sequence as shown in SEQ ID NO: 3 and a primer consisting of the nucleotide sequence as shown in SEQ ID NO: 4 may be used. As a primer used for isolating a region equivalent to the Klenow fragment, a primer consisting of the nucleotide sequence as shown in SEQ ID NO: 5 may be used. Microorganisms are not limited, and a wide variety of bacteria can be used in addition to the Bacillus organisms. The pol I type DNA polymerase may be isolated from a microorganism by preparing a pair of primers corresponding to the sequences of the conserved regions, preparing a genomic DNA template from the microorganism, amplifying the genomic DNA template using such primers, cloning the amplified fragment, amplifying the pol I type DNA polymerase gene using a pair or primers that sandwich a region equivalent to the ORF or Klenow fragment, constructing a pol I type DNA polymerase expression plasmid, and allowing the gene to express.

The present invention also includes the pol I type DNA polymerase derived from the microorganism thus obtained and DNA that encodes such polymerase.

The poly I type polymerase of the present invention has common activity of an enzyme for template-dependent DNA replication, activity of an enzyme for complementary strand-displacement replication, and reverse transcriptase activity. Also, such polymerase may have 3′→5′ exonuclease activity. An example of an advantage of the presence of 3′→5′ exonuclease activity is a lower frequency of errors that occur at the time of substrate incorporation. The optimal temperature of the pol I type DNA polymerase of the present invention is lower than that of the DNA polymerase having known activity of an enzyme for complementary strand-displacement replication. The polymerase of the present invention exhibits its activity at 50° C. to 60° C., and preferably 50° C. to 55° C. The DNA polymerase of the present invention can be accordingly used at a reaction temperature lower than that of the DNA polymerase having conventional activity of an enzyme for complementary strand-displacement replication; i.e., such polymerase can be used at a temperature closer to the room temperature. Because of reverse transcriptase activity, DNA can be synthesized from the RNA template, and it can be used for a technique alternative to conventional RT-PCR.

The present invention includes a protein that is the pol I type DNA polymerase obtained in the aforementioned manner and DNA that encodes such polymerase.

SEQ ID NO: 6 shows the nucleotide sequence of DNA that encodes the pol I type DNA polymerase of the present invention, and SEQ ID NO: 7 shows the amino acid sequence of the pol I type DNA polymerase of the present invention.

Further, the present invention include a ΔN pol I type DNA polymerase that lacks the amino acids of N-terminal region of the pol I type DNA polymerase and DNA that encodes the same. The number of the amino acid residues of the N-terminal region to be deleted is not limited, provided that the ΔN pol I type DNA polymerase has activity of an enzyme for complementary strand-displacement replication. Preferably, the ΔN poly type DNA polymerase lacks 5′→3′ exonuclease activity and has activity equivalent to that of the Klenow fragment of E. coli DNA polymerase I. The ΔN pol I type DNA polymerase lacks a consecutive amino acid sequence from 1st Val to any amino acid up to 297th Glu of the pol I type DNA polymerase as shown in SEQ ID NO: 7. The ΔN pol I type DNA polymerase equivalent to the Klenow fragment lacks 297 amino acid residues from Val-1 up to Glu-297 of the amino acid sequence of the pol I type DNA polymerase as shown in SEQ ID NO: 7. SEQ ID NO: 8 shows the DNA sequence that encodes the ΔN pol I type DNA polymerase equivalent to the Klenow fragment, and SEQ ID NO: 9 shows the amino acid sequence of the ΔN pol I type DNA polymerase equivalent to the Klenow fragment. More specifically, the protein of the present invention includes a protein that lacks a consecutive amino acid sequence from 1st Val to any amino acid up to 297th Glu of the amino acid sequence as shown in SEQ ID NO: 7 and has activity of an enzyme for complementary strand-displacement replication. When the number of amino acid residues that are not present is large, such protein lacks 5′→3′ exonuclease activity. In such a case, advantageously, the amplified product would not be degraded at the time of gene amplification.

Further, the present invention includes a protein that is the pol I type DNA polymerase and lacks 3′→5′ exonuclease activity and DNA that encodes such protein. The pol I type DNA polymerase having 3′→5′ exonuclease activity disadvantageously degrades the 3′ region of the primer, and accordingly, the rate of nucleic acid amplification becomes slower. In contrast, the pol I type DNA polymerase lacking 3′→5′ exonuclease activity does not degrade the primer and accordingly, the rate of nucleic acid amplification advantageously becomes faster. If the pol I type DNA polymerase having 3′→5′ exonuclease activity is used for detecting DNA mutation, the 3′ region of the primer is degraded, the site of mutation cannot be recognized by the primer, the extension synthesis reaction proceeds disadvantageously, and mutation cannot be detected. If the pol I type DNA polymerase lacking 3′→5′ exonuclease activity is used, however, the primers are not degraded, the primers are not annealed at the site of mutation, the extension synthesis reaction can be terminated, and mutation can thus be advantageously detected.

The present invention also includes DNA that can hybridize under stringent conditions to DNA consisting of a sequence complementary to the DNA sequence as shown in SEQ ID NO: 6 and that encodes a protein having activity of the pol I type DNA polymerase. Also, the gene of the present invention includes DNA that can hybridize under stringent conditions to DNA consisting of a sequence complementary to the DNA sequence as shown in SEQ ID NO: 8 and encodes a protein having activity of the pol I type DNA polymerase but lacking 5′→3′ exonuclease activity. Under stringent conditions, for example, hybridization can be carried out with the use of a filter on which DNA has been immobilized in the presence of 0.7 to 1.0 M NaCl at 68° C., the filter may be washed with a 0.1- to 2-fold SSC solution (1-fold SSC consists of 150 mM of NaCl and 15 mM of sodium citrate) at 68° C., and DNA can then be identified.

DNA that encodes the pol I type DNA polymerase of the present invention includes DNA consisting of the nucleotide sequence that encodes a protein satisfying the following conditions. That is, when calculating the homology using BLAST or other means under default conditions, DNA consists of the nucleotide sequence that encodes a protein having 80% or higher, preferably 90% or higher, and more preferably 95% or higher homology with the nucleotide sequence as shown in SEQ ID NO: 6 and has activity of the pol I type DNA polymerase, and DNA consists of the nucleotide sequence that encodes a protein having 80% or higher, preferably 90% or higher, and more preferably 95% or higher homology with the nucleotide sequence as shown in SEQ ID NO: 8, has activity of the pol I type DNA polymerase, but lacks 5′→3′ exonuclease activity. Further, the present invention includes RNA that reacts with the above DNA or RNA that can hybridize under stringent conditions to the aforementioned RNA having activity of the pol I type DNA polymerase or having activity of the pol I type DNA polymerase but lacking 5′→3′ exonuclease activity.

The DNA of the present invention further includes a degenerate mutant of the nucleotide sequence as shown in SEQ ID NO: 6 or 8.

Mutation can be introduced into a gene by conventional techniques such as the Kunkel method or the Gapped duplex method or a method in accordance therewith. For example, a mutagenesis kit (e.g., Mutant-K (TAKARA) or Mutant-G (TAKARA)) that utilizes site-directed mutagenesis can be used to easily introduce mutation.

Further, the present invention includes a protein consisting of an amino acid sequence derived from the amino acid sequence as shown in SEQ ID NO: 7 by mutation such as deletion, substitution, or addition of one or several amino acid residues, which has activity of the pol I type DNA polymerase and a protein consisting of an amino acid sequence derived from the amino acid sequence as shown in SEQ ID NO: 9 by mutation such as deletion, substitution, or addition of one or several amino acid residues, which has activity of the pol I type DNA polymerase but lacks 5′→3′ exonuclease activity. Further, the present invention includes a protein consisting of an amino acid sequence derived by mutation, such as deletion, substitution, or addition of one or several amino acid residues, from the amino acid sequence derived from the amino acid sequence as shown in SEQ ID NO: 7 by deletion of a consecutive amino acid sequence from 1st Val to any amino acid up to 297th Glu, which has activity of the pol I type DNA polymerase. Such protein may lack or maintain 5′→3′ exonuclease activity. “Deletion, substitution, or addition of one or several amino acid residues” refers to deletion, substitution, or addition of 1 to 10, preferably 1 to 5, and more preferably 1 or 2 amino acid residues.

Further, the present invention includes DNA that encodes a protein consisting of the amino acid sequence shown above.

The present invention includes a primer and a probe that are used for isolating a DNA polymerase from a microorganism. Such primer or probe is a fragment of the above DNA, and the number of nucleotides is 5 to 50, preferably 10 to 30, and more preferably 15 to 25. The length of the nucleotide sequence to be amplified is not limited.

DNA that encodes the pol I type DNA polymerase of the present invention and a mutant thereof are inserted into an expression vector, the expression vector is introduced into an adequate host cell, and such host cell is cultured. Thus, pol I type DNA polymerase can be obtained. In such a case, DNA encoding GST or DNA encoding hexahistidine may be adequately ligated. Any vector can be used, provided that such vector is capable of replicating the gene of interest in a host cell, such as a plasmid, phage, or virus host. Examples include E. coli plasmids such as pBR322, pBR325, pUC118, pUC119, pKC30, and pCFM536, Bacillus subtilis plasmids such as pUB110, yeast plasmids such as pG-1, YEp13, and YCp50, and DNAs of phages such as λgt110 and λZAPII. Examples of mammalian cell expression vectors include virus DNA such as that of a baculovirus, vaccinia virus, or adenovirus, SV40, and derivatives thereof. A vector comprises the replication origin, a selection marker, and a promoter. According to need, a vector may further comprise an enhancer, a terminator, a ribosome binding site, a polyadenylation signal, and the like. Any promoter can be used, provided that expression is efficient in a host cells. Examples thereof include an SRα promoter, SV40 promoter, LTR promoter, CMV promoter, and HSV-TK promoter.

Examples of host cells include bacterial cells, such as E. coli, Streptomyces, or Bacillus subtilis, fungal cells, such as those of the Aspergillus strain, yeast cells, such as bread yeast and methanol-assimilable yeast, insect cells such as those of drosophila S2 or Spodoptera Sf9, and mammalian cells, such as HEK293T, HeLa, SH-SY5Y, CHO, COS, BHK, 3T3, and C127 cells.

Transformation can be carried out by conventional techniques such as the calcium chloride method, the calcium phosphate method, the DEAE-dextran-mediated transfection method, or electroporation.

The pol I type DNA polymerase can be isolated and purified by a common biochemical method used for isolation and purification of a protein, such as ammonium sulfate precipitation, gel chromatography, ion-exchange chromatography, or affinity chromatography. Such technique may be employed alone or in adequate combinations.

The present invention includes an antibody that reacts with the pol I type DNA polymerase. The antibody may be a polyclonal or monoclonal antibody. The antibody can be prepared by a known technique. The antibody comprises a functional fragment, and the term “functional fragment” used with reference to an antibody refers to any molecule that carries a variable region of an antibody molecule containing a (Fab)₂ fragment, (Fab) fragment, and the like. The resulting antibody can be used for purifying the pol I type DNA polymerase of the present invention, for example.

Further, the present invention includes a method for nucleic acid amplification using the pol I type DNA polymerase of the present invention. Examples of methods for nucleic acid amplification include conventional PCR (polymerase chain reaction method), RT-PCR (the reverse transcriptase polymerase chain reaction method), the Mitani method (WO 01/030993), the LAMP (the loop-mediated isothermal amplification) method (Nucleic Acids Res 28, No. 12, e63, 2000; Igaku no ayumi (progress of medicine), Vol. 206, No. 8, 470-474, 2003; Molecular and Cellular Probes, Vol. 16, No. 3, 223-229, 2002), the SDA (the strand displacement amplification) method (JP Patent Publication (kokai) No. 10-313900 A (1998); “Kensa to gijutsu (Test and technique),” Vol. 24, No. 3, 1996, Takashi Sato, Igaku shoin, pages 241 to 243, the ICAN® (isothermal and chimeric primer-initiated amplification of nucleic acids) method (WO 00/56877), the TRC (transcription reverse transcription concerted reaction) method, and the NASBA (nucleic acid sequence based amplification) method (JP Patent Law No. 2,650,159). The pol I type DNA polymerase of the present invention can be used in any such techniques. It is particularly suitable for isothermal amplification methods, such as the Mitani method, the LAMP method, the SDA method, or ICAN®. In such gene amplification techniques, the pol I type DNA polymerase of the present invention can be used instead of DNA polymerases that have been used in the past.

Further, the present invention includes a kit for nucleic acid amplification using the pol I type DNA polymerase of the present invention. This kit is suitable for any of the aforementioned techniques for nucleic acid amplification. The kit comprises primers, dNTP, Tris-HCl, HCl, MgSO₄, betaine, and the like, in addition to the pol I type DNA polymerase of the present invention.

The present invention is described in greater detail with reference to the following examples, although the technical scope of the present invention is not limited thereto.

1. Cloning of Bacillus smithii DNA Polymerase

Genomic DNA was prepared from a cultured B. smithii JCM9076 cell by a conventional technique. Since phoR and mutM are conserved in regions upstream and downstream of the pol A gene construct of the other Bacillus species, primers PhoF (SEQ ID NO: 1) and MutR (SEQ ID NO: 2) were then designed from the conserved regions of the relevant genes. Further, PCR was carried out using the prepared genomic DNA as a template and PhoF and MutR as primers to amplify the gene, the amplified fragment was cloned using pGEM-T (Promega), and the internal sequence was determined by a conventional technique. A putative pol A ORF region was amplified by PCR from the determined sequence using Bsth-EcoNF (SEQ ID NO: 3) and Bsth-SalCR (SEQ ID NO: 4). The resulting PCR product and pUC18 were digested with EcoRI and SalI, and they were ligated to each other by mixing to construct a Bsm DNA polymerase I expression plasmid. From the thus-determined sequence, a fragment was amplified by PCR using Bsth-EcoLF (SEQ ID NO: 5) and Bsth-SalCR (SEQ ID NO: 4), which are regions of pol A equivalent to the N-terminus of the E. coli Klenow fragment. The resulting PCR product and pUC18 were digested with EcoRI and SalI, and they were ligated to each other by mixing to construct a ΔN Bsm DNA polymerase expression plasmid.

### 2. Expression and Purification of Bsm DNA Polymerase I and ΔN Bsm DNA Polymerase I

### (1) Culture, Expression, and Preparation of Crude Extract

E. coli XL1-Blue comprising pUCBsm or pUCdNBsm was cultured in 5 ml of LB medium containing 100 μg/ml of ampicillin at 37° C. overnight to prepare a preculture solution. The resulting preculture solution (5 ml) was sowed in 500 ml of LB medium containing 100 μg/ml of ampicillin, and shake culture was carried out at 37° C. and 200 rpm (Orbital Shaking Incubator, FIRSTEK OSI-502LD). When the OD value at 600 nm reached around 0.5, 1 mM of IPTG was added. Shake culture was further carried out at 37° C. and 200 rpm for 1 to 2 hours. The resulting culture solution was transferred to a centrifugation tube, and centrifugation was carried out at 4,000×g for 10 minutes to obtain a precipitate. The obtained precipitate was suspended in 30 ml of 1×PBS, centrifugation was carried out again at 4,000×g for 10 minutes, and cells were washed. The obtained precipitate was suspended in 25 ml of 1×PBS, and cells were disrupted by ultrasonic irradiation (MISONIX Astrason ultrasonic processor XL) for 10 seconds 6 times. The ultrasonically disrupted samples were centrifuged at 15,000×g for 30 minutes to obtain a supernatant. A 30% polyethyleneimine solution was added to a final concentration of 0.1% in the supernatant, the resulting mixture was allowed to stand on ice for 30 minutes, and the resultant was centrifuged at 15,000×g for 30 minutes to obtain a supernatant. This supernatant was designated as a crude extract.

### (2) Anion Exchange Column Chromatography

Ion exchange chromatography was carried out using the AKTA Prime high-performance liquid chromatography system (GE Healthcare) and a strong anion exchange column (HiTrap Q, GE Healthcare). A running buffer comprising 50 mM Tris-HCl (pH 7.6), 2 mM EDTA, and 10 mM 2-mercaptoethanol was used. The column was equilibrated at a flow rate of 1 ml/min, the crude extract was applied thereto, and the nonadsorption fraction was washed with the same running buffer. The adsorption fraction was eluted with about 15 CV at a sodium chloride concentration gradient of from 0 to 1M. The elution fraction was fractionated to result in 1-ml-each fractions, the fractions were subjected to SDS-PAGE to observe the protein band of the relevant molecular weight, and a fraction containing such band was recovered. The recovered fraction was concentrated and desalted using a ultrafiltration membrane, and the obtained fraction was designated as an “anion exchange fraction.”

### (3) Heparin Affinity Column Chromatography

Heparin affinity column chromatography was carried out using the AKTA Prime high-performance liquid chromatography system (GE Healthcare) and a heparin affinity column (HiTrap Heparin, GE Healthcare). The solution used in anion exchange column chromatography was used as the running buffer. The column was equilibrated at a flow rate of 1 ml/min, the anion exchange fraction was applied, and the nonadsorption fraction was washed with the same running buffer. The adsorption fraction was eluted with about 22 CV at a sodium chloride concentration gradient of 0 to 1M. The elution fraction was fractionated to result in 1-ml-each fractions, the fractions were subjected to SDS-PAGE to observe the protein band having such molecular weight, and a fraction comprising such band was recovered. The recovered fraction was subjected to buffer exchange using an ultrafiltration membrane with 50 mM Tris-HCl (pH 8.0) and 0.2M sodium chloride, the product was further concentrated, and the resultant was designated as the “heparin fraction.”

### (4) Gel Filtration Column Chromatography

Gel filtration column chromatography was carried out using the AKTA 10XT high-performance liquid chromatography system (GE Healthcare) and the gel filtration column (HiLoad 16/60 Superdex 200 prep grade, GE Healthcare). A running buffer comprising 50 mM Tris-HCl (pH 8.0) and 0.2M sodium chloride was used. The column was equilibrated at a flow rate of 1 ml/min, and the heparin fraction was applied, followed by elution with the same running buffer. The elution fraction was fractionated to result in 1-ml-each fractions, the fractions were subjected to SDA-PAGE to observe the protein band of the relevant molecular weight, and a fraction comprising such band was recovered. The recovered fraction was concentrated using an ultrafiltration membrane, the buffer was exchanged with a stock buffer (50 mM potassium chloride, 10 mM Tris-HCl (pH 7.5), 1 mM DTT, 0.1 mM EDTA, 0.1% Triton X-100, and 50% glycerol), and the resultant was designated as a purified enzyme sample.

### 3. Assay of DNA Polymerase Activity

DNA polymerase activity was assayed using the Picogreen dsDNA quantitation reagent (Invitrogen) with reference to Biotechniques 21, 664-668, 1996. The Picogreen dsDNA quantitation reagent was mixed with a TE buffer at a ratio of 1:345, 173 μl of the resulting mixture was added to 27 μl of a mixture of M13mp18 single-stranded DNA, primers, dNTP, and the purified enzyme sample, the resultant was allowed to stand at room temperature for 5 minutes, and fluorescence intensity was assayed at an excitation wavelength of 480 nm and an assay wavelength of 520 nm. The commercially available Klenow fragment of a known unit was also assayed in the same manner, and an enzyme unit was determined based thereon as a relative value. Examples of assay results are shown below. Standard lines were prepared for each assay. Using the commercially available Bst DNA polymerase as a standard, the fluorescence intensity at various dilution ratios was assayed using the Picogreen dsDNA quantitation reagent. The results as shown in Table 1 were obtained.

TABLE 1

| Bst DNA polymerase | Measurement 1 | Measurement 2 |
|---|---|---|
| 0.5 units | 57.699 | 61.725 |
| 1.0 unit | 60.198 | 63.044 |
| 2.0 units | 76.175 | 88.095 |
| 4.0 units | 93.177 | 92.283 |
| 6.0 units | 93.427 | 105.38 |
| 8.0 units | 112.78 | 132.35 |

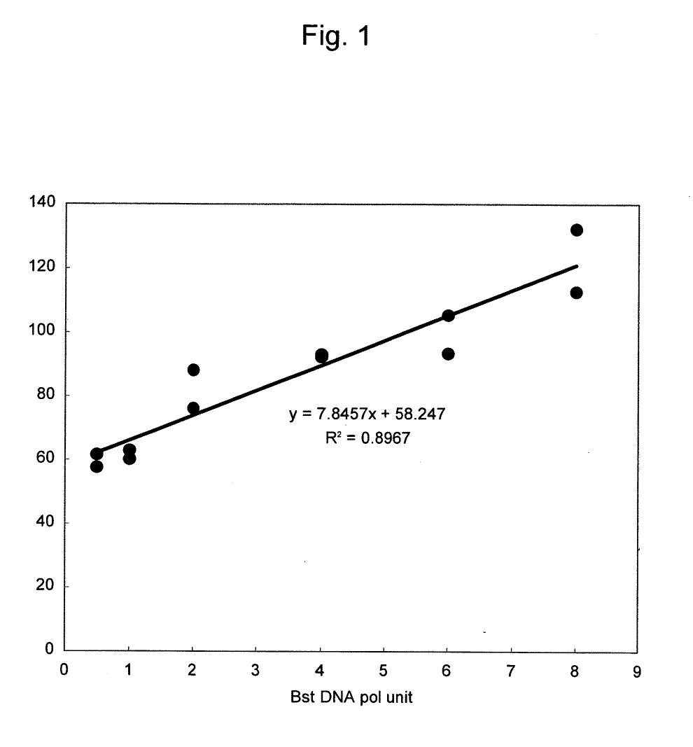

The results were plotted and regressed to the primary linear line. Consequently, the equation shown below was obtained. FIG. 1 shows the regression line.

y=7.8457x+58.247(R²=0.8967)

The Bsm DNA polymerase samples that had been assayed simultaneously exhibited a fluorescence intensity of 96.26 on average (first result: 98.814; second result: 93.707). Accordingly, it was calculated to be about 4.85 units based on this regression equation.

### 4. Assay of Activity for Complementary Strand-Displacement Replication

In accordance with Nucleic Acids Res 28, No. 12, e63, 2000, the LAMP method was carried out by allowing 20 μl of a mixture of synthetic DNAs of M13mp18 single-stranded DNA, 0.8 μM FIP, 0.8 μM BIP, 0.2 μM F3, and 0.2 μM B3, 1M betaine, 20 mM Tris-HCl buffer (pH 8.8), 10 mM potassium chloride, 10 mM ammonium sulfate, 0.1% Triton X-100, and 2 to 4 mM magnesium sulfate to stand at 95° C. for 5 minutes and then on ice for 5 minutes. The purified enzyme sample (5 μl) was added thereto, the resulting mixture was allowed to stand at 55° C. to 65° C. for 1 hour, and the resultant was subjected to agarose gel electrophoresis. A commercially available Bst DNA polymerase (ew England Biolabs, No. M0275S) of a known unit was also assayed in the same manner, the density of the band of the electrophoresed product was determined, and the enzyme unit was determined based thereon as a relative value.

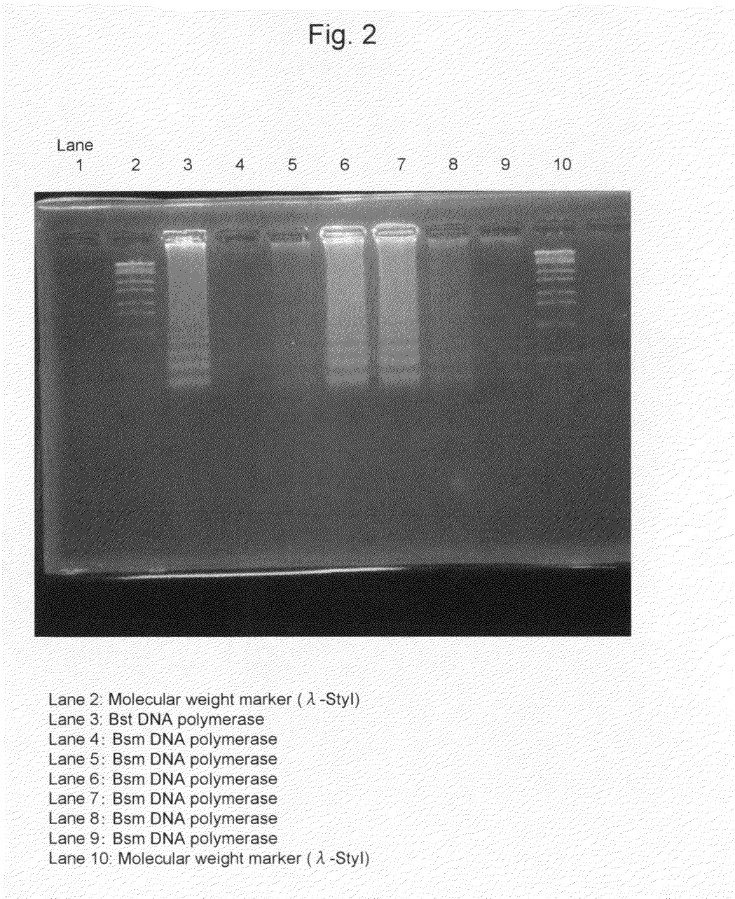

The results are shown in FIG. 2. As shown in FIG. 2, the results indicated in lane 6 and in lane 7 were substantially the same as those for the commercially available Bst DNA polymerase of a known unit (lane 3 of FIG. 2). This indicates that 5 μl of the enzyme samples used for lane 6 and lane 7 had activity equivalent to that of the Bst DNA polymerase of a known unit.

### 5. Reverse Transcriptase Activity

Reverse transcriptase activity was assayed using the EnzChek reverse transcriptase assay kit (Invitrogen) in accordance with the instructions, and the reaction product was detected. Also, reverse transcription was carried out using an RNA ladder (0.24 to 9.5 kb, Invitrogen) as the template, the reaction product was subjected to agarose gel electrophoresis, and the reaction product was detected via autoradiography.

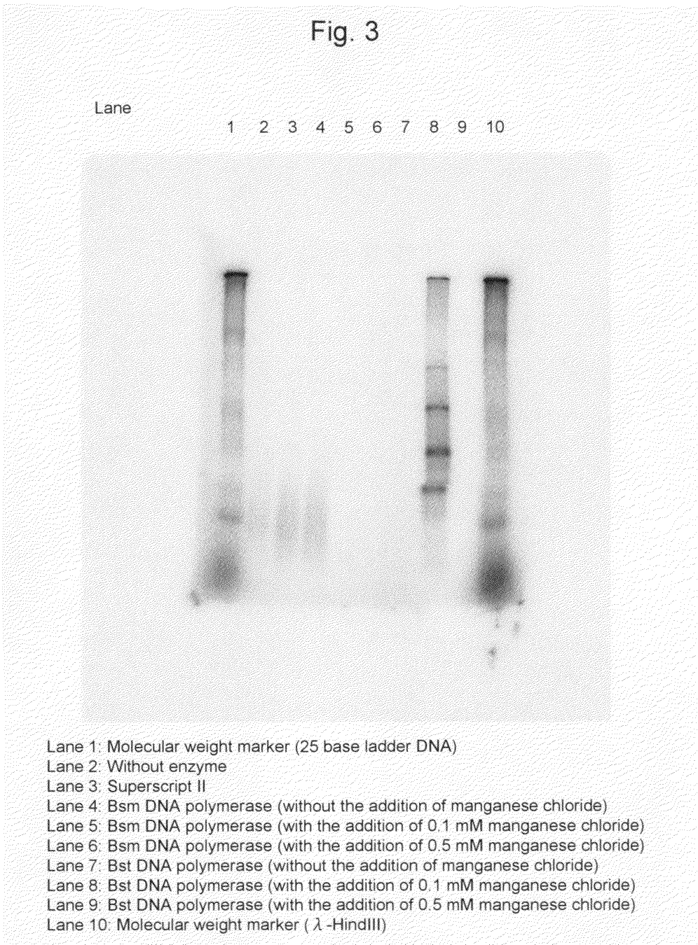

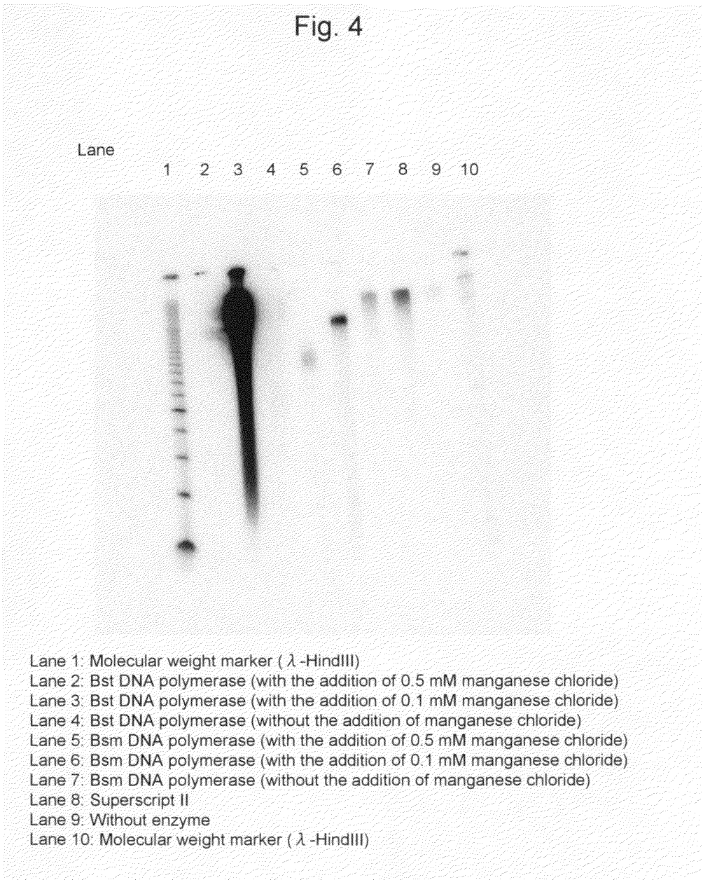

The results are shown in FIG. 3 and in FIG. 4. As shown in lanes 4 to 6 of FIG. 3, a reverse transcript was detected regardless of the presence or absence of manganese chloride. This reverse transcript was longer than the reverse transcript of the Bst DNA polymerase, as shown in lanes 2 to 4 and lanes 5 to 6 of FIG. 4.

### 6. Isothermal Gene Amplification

Isothermal gene amplification was carried out under the following conditions.

Amplification conditions: 60° C. for 90 minutes

Reaction solution (in 25 μl): Tris-HCl (20 mM, pH 8.8), KCl (10 mM), (NH₄)₂SO₄ (10 mM), SYBR green (0.01 μl/ml), 8 mM MgSO₄, 0.1% Tween 20, 0.5M betaine, 1.4 mM dNTP, 4 units of Bsm or Bst DNA polymerase, and 40 ng of human genomic DNA

The following primers were used.

| | |
|---|---|
| 1,600 nM of EF5 | |
| ACAACGAGGCGCAGCAGAGGGGACATGAAA | (SEQ ID NO: 11) |
| 1,600 nM of ER6 | |
| TTGAAGACGTAAAGACTCTTTCACATCCTC | (SEQ ID NO: 12) |
| 8 mM of ER6-L1 | |
| TGTGCCATTCCAAAGG | (SEQ ID NO: 13) |

Gene amplification was monitored on a real-time basis in a reaction solution using Mx3000P (Stratagene) in the presence of SYBR green I (Molecular Probes) at 60° C. for 90 minutes.

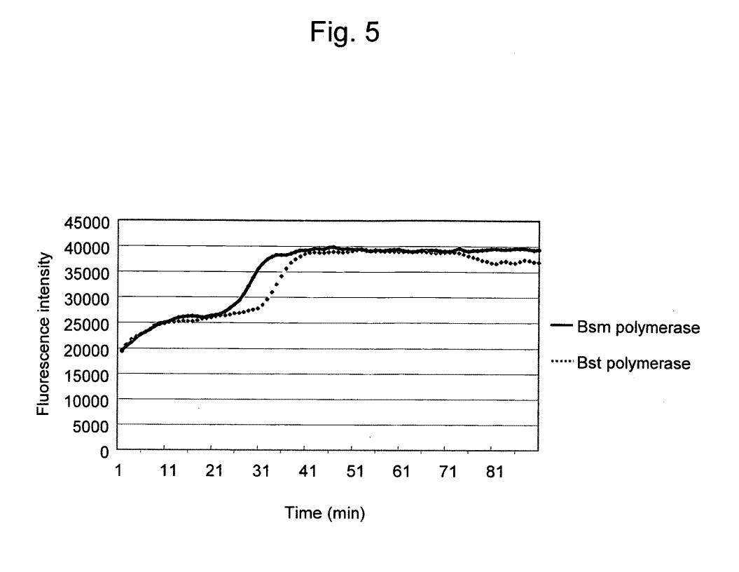

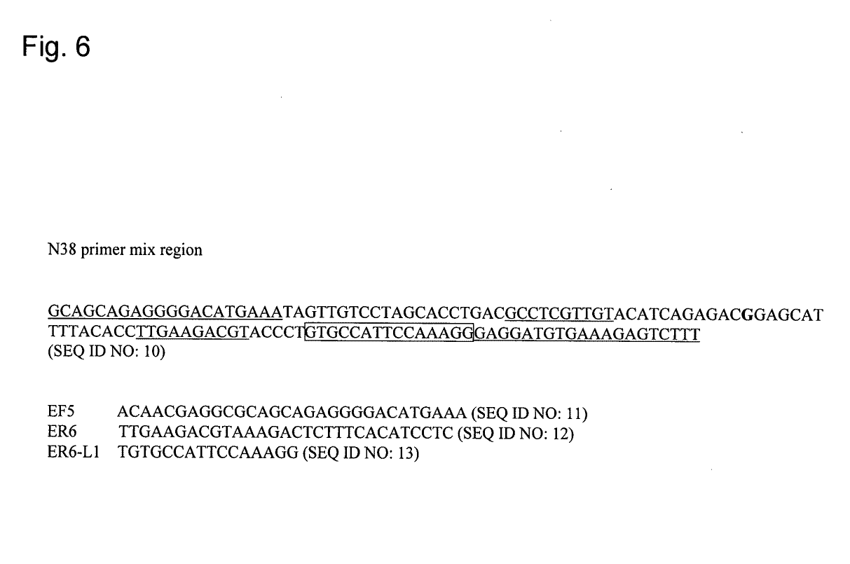

The results are shown in FIG. 5. When the Bsm DNA polymerase of the present invention was used, amplification proceeded faster than amplification with the use of a conventional Bst DNA polymerase, as shown in FIG. 5. The amplified sequence was read using a sequencer in order to confirm that the target sequence had been amplified. As a result, the target sequence was found to be amplified. FIG. 6 shows the positional relationship between the template sequence (SEQ ID NO: 10) and the primer. In the sequence shown in FIG. 6, underlined regions indicate primer regions, and a loop primer is surrounded with a frame.

### INDUSTRIAL APPLICABILITY

The DNA polymerase of the present invention exhibits activity at a lower temperature than conventional DNA polymerase having strand-displacement activity. Accordingly, the DNA polymerase of the present invention can be used at a temperature closer to room temperature. Since the DNA polymerase of the present invention has reverse transcriptase activity, DNA can be synthesized from an RNA template, and it can be used for a technique that serves as an alternative to conventional RT-PCR.

All publications, patents, and patent applications cited herein are incorporated herein by reference in their entirety.

### Sequence Listing Free Text

SEQ ID NOs: 1 to 5 and 10 to 13: synthetic sequences

1. Pol I type DNA polymerase, which is any of proteins (a) to (f) below and has DNA polymerase activity:
(a) a protein comprising the amino acid sequence as shown in SEQ ID NO: 7;
(b) a protein consisting of an amino acid sequence derived from the amino acid sequence as shown in SEQ ID NO: 7 by deletion, substitution, or addition of one or several amino acid residues;
(c) a protein consisting of the amino acid sequence as shown in SEQ ID NO: 9;
(d) a protein consisting of an amino acid sequence derived from the amino acid sequence as shown in SEQ ID NO: 9 by deletion, substitution, or addition of one or several amino acid residues;
(e) a protein consisting of an amino acid sequence derived from the amino acid sequence as shown in SEQ ID NO: 7 by deletion of a consecutive amino acid sequence from 1st Val to any amino acid up to 297th Glu; and
(f) a protein consisting of an amino acid sequence derived by deletion, substitution, or addition of one or several amino acid residues from the amino acid sequence derived from the amino acid sequence as shown in SEQ ID NO: 7 by deletion of a consecutive amino acid sequence from 1st Val to any amino acid up to 297th Glu.

2. DNA encoding any of proteins (a) to (f) below having DNA polymerase activity:
(a) a protein comprising the amino acid sequence as shown in SEQ ID NO: 7;
(b) a protein consisting of an amino acid sequence derived from the amino acid sequence as shown in SEQ ID NO: 7 by deletion, substitution, or addition of one or several amino acid residues;
(c) a protein consisting of the amino acid sequence as shown in SEQ ID NO: 9;
(d) a protein consisting of an amino acid sequence derived from the amino acid sequence as shown in SEQ ID NO: 9 by deletion, substitution, or addition of one or several amino acid residues;
(e) a protein consisting of an amino acid sequence derived from the amino acid sequence as shown in SEQ ID NO: 7 by deletion of a consecutive amino acid sequence from 1st Val to any amino acid up to 297th Glu; and
(f) a protein consisting of an amino acid sequence derived by deletion, substitution, or addition of one or several amino acid residues from the amino acid sequence derived from the amino acid sequence as shown in SEQ ID NO: 7 by deletion of a consecutive amino acid sequence from 1st Val to any amino acid up to 297th Glu.

3. DNA, which is any of (g) to (j) below and encodes a protein having DNA polymerase activity:
(g) DNA consisting of the nucleotide sequence as shown in SEQ ID NO: 6;
(h) DNA hybridizing under stringent conditions to DNA consisting of a sequence complementary to DNA consisting of the nucleotide sequence as shown in SEQ ID NO: 6 and encoding a protein;
(i) DNA consisting of the nucleotide sequence as shown in SEQ ID NO: 8; and
(j) DNA hybridizing under stringent conditions to DNA consisting of a sequence complementary to DNA consisting of the nucleotide sequence as shown in SEQ ID NO: 8 and encoding a protein.

4. A recombinant vector comprising DNA according to claim 2.

5. A transformant comprising the recombinant vector according to claim 4.

6. A method for producing a pol I type DNA polymerase which comprises culturing the transformant according to claim 5 and sampling the pol I type DNA polymerase from the culture product.

7. The pol I type DNA polymerase according to claim 1 having activity of an enzyme for complementary strand-displacement replication and reverse transcriptase activity.

8. The pol I type DNA polymerase according to claim 1 lacking 5′→3′ exonuclease activity.

9. The pol I type DNA polymerase according to claim 1 having 3′→5′ exonuclease activity.

10. The pol I type DNA polymerase according to claim 1 lacking 3′→5′ exonuclease activity.

11. A method for nucleic acid amplification using the pol I type DNA polymerase according to claim 1.

12. The method for nucleic acid amplification according to claim 11, which is an isothermal amplification method.

13. A kit for nucleic acid amplification comprising the pol I type DNA polymerase according to claim 1.

14. A method for cloning the pol I type DNA polymerase gene comprising steps of:
(1) preparing a primer consisting of the nucleotide sequence as shown in SEQ ID NO: 1 and a primer consisting of the nucleotide sequence as shown in SEQ ID NO: 2;
(2) preparing a genomic DNA template;
(3) amplifying a genomic DNA template using a primer consisting of the nucleotide sequence as shown in SEQ ID NO: 1 and a primer consisting of the nucleotide sequence as shown in SEQ ID NO: 2;
(4) cloning the amplified fragment of (3);
(5) amplifying DNA encoding pol I type DNA polymerase using a primer consisting of the nucleotide sequence as shown in SEQ ID NO: 3 and a primer consisting of the nucleotide sequence as shown in SEQ ID NO: 4; and
(6) cloning the amplified fragment of (5).

15. A method for cloning the pol I type DNA polymerase gene consisting of the sequence as shown in SEQ ID NO: 8 which comprises steps of:
(1) preparing a primer consisting of the nucleotide sequence as shown in SEQ ID NO: 4 and a primer consisting of the nucleotide sequence as shown in SEQ ID NO: 5;
(2) preparing a genomic DNA template;
(3) amplifying a genomic DNA template using a primer consisting of the nucleotide sequence as shown in SEQ ID NO: 4 and a primer consisting of the nucleotide sequence as shown in SEQ ID NO: 5; and
(4) cloning the amplified fragment.

16. A primer consisting of a fragment of the DNA according to claim 2 and comprising 5 to 50 nucleotides.

17. A recombinant vector comprising DNA according to claim 3.

18. A primer consisting of a fragment of the DNA according to claim 3 and comprising 5 to 50 nucleotides.


SEQUENCE LISTING (13 sequences)

SEQ ID NO: 1 - DNA, 22 bp, Artificial Sequence
  9, 18 and 21: N is A, G, C or T
tttgarcgnttttaymgngtng

SEQ ID NO: 2 - DNA, 21 bp, Artificial Sequence
  4, 10 and 19: N is A, G, C or T
ytcnacytcnggyaaytcngg

SEQ ID NO: 3 - DNA, 31 bp, Artificial Sequence
  Description of Artificial Sequence Synthetic
acaaaggggaattccctagtgaagaagaaac

SEQ ID NO: 4 - DNA, 32 bp, Artificial Sequence
  Description of Artificial Sequence Synthetic
aaaacctatcacctccagtcgacctatttagc

SEQ ID NO: 5 - DNA, 33 bp, Artificial Sequence
  Description of Artificial Sequence Synthetic
gaaacagaagtgaattcattaatgaaattggag

SEQ ID NO: 6 - DNA, 2908 bp, Bacillus smithii
  CDS (254)..(2884)
ccgcttatcgccctctgatagatctctccgcttgaaatcagctcattgaatccaattggaaaacaagcttttgtcatataaaatagttttgttaaatggtggaacgtccttggtgcccgattagtgagaaatggcgcattttcatgattgaaggggcctccataaagaaaaagcggatggaaaaccgcttttttcttttcggttctttgcatggtagaataacaagtaggagaagacaaagggaggttgcctagtgaagaagaaactgattttgatagatggaaacaacattgcataccgtgcttttttcgcccttccgttattaaataatgaaaaaggtattcacaccaatgcgatttacggctttacgatgatgctgaacaaaatattagaggaagaaaagccaacccatatgttggtggcatttgatgccggcaaaacgacttttcgacatgaaacattcaaggaatataaaggcggcaggcaaaaaacgcctccggaactatcggagcaatttccgtttatccgcgatttattaaagtcttttcacattccgcaatttgaactggaaaattatgaagcggatgatatcattggtaccctttcattagaagcggagaaaaaagattttgaaatcaaaatttacagcggagacaaagatttgacccaattggcttcggagaaaacaacggtttgtctctgtcgaaagggaattaccgatattgaagaatacactccggaacatgtaaaagaaaaatacggtcttacccctcaacaaattatcgacatgaaaggtttgatgggagactcttcggataatattcctggagtccctggaatcggcgaaaaaacggcgattaaactgttaaaagaatttgaaaccgttgaaaaggtagtcgattctattgatgaaatcagcggaaaaaagctaaaagaacggcttcaagaacataaacaacaagctttaatgagcaaagaactagcaaccattaaaagagatgctcctttagggattacggttgacgaacttgagtatcaaggaccggactgggaaaaagttcagagtatttataaggaacttggattccagtctctgctggagaaaatagatcaaactcaggaaacagaagtgcagccattagaaaaattggagtatcaaactgtcgaagaaatcacggaggatttgttcgaacaggaaaattctttttatttagagatgattggtgaaaattatttcattagcgacattgtcggtatggctgttcaaaatgaaaaaggaattttttatttgccgacagaatcaactttaaaatctcctgtttttaaaaaatgggctgaagacgaaacgaaaaagaaaacggtgtttgatgctaagcgtacggttattgccttgcggaggcatggaattgaactgaaagggattgaatttgatttactgctagcttcttatttaattaacccgtctgaatccccttccgattttgccgatgtagctaagcttcatggttttaatgaagtgcaatccgacgaagcggtctacggaaaaggggcgaaattaaagctcccggacagagaaatttatgaggagcatattgcaagaaaagcggtggggcttactaaattggcggaaacatgtcgagatgttctgaaagaaaatgatcagctctctcttttttacgatttagaaatgccgcttgctttgattttggccgatatggaatggacaggcgtaaaggtggacgtggatcggttgacagaaatgggtgacgaacttcacaacaggctgcaggaaatcgaaaaggaaatctatgaattggccggacaagagtttaacattaactctcctaaacaactagggcatattttatttgaaaaaatggggctgccggtgataaagaaaacgaaaacaggttattccacatccgccgatgtattggaaaaattggaaagcagtcatgaaatcgttcgctacattttggagtaccgccagctaggaaaattgcaatccacttatatagatgggctgttgaaagtcgttcaccaaaatactcataaagtgcatactcgctttaatcaagctcttacacagacaggcagactgagttccgcagatcctaatttgcaaaacatcccgatcagactcgaagaaggaaggaaaatccgccaagcttttgtcccgtctgaaaaagattgggtcatcttttcagcggattattcacaaatcgaattgcgggttctcgctcatatatccggtgatcaaaagcttattgaagccttccgagaggatatggatatccacaccaaaacggccatggatgtttttcatgtacaaaaagaagaagtgacatccaatatgaggagacaggcgaaagccgttaattttgggattgtctatggaatcagcgattatggactctcacaaaatttagggattacgagaaaggaagccggtcagtttattgaacgttattttgcttcttatccggatgtaaaagaatatatggatgagattgttagagaagcgaaacgaaaaggttatgtaaccacattgcttcatagaagacgatatttgccggagattacaagccgaaatttcaatgtacgcagctttgcagagcggacggccatgaatacgccaatacaaggaagcgctgctgatattattaaaaaagcaatgattgatatggcagaacgattgaagaaagaacagcttaaatcgagaatgcttcttcaagtgcatgatgaattgatttttgaagttcctcccgatgagatagaaacgatgaaaaaaatcgtaccagatgtaatggaacatgcggttgaattgaaagttccgctaaaagtggattatgcctatggccccacttggtatgatgctaaataggcggtttggaggtgataggttttg

SEQ ID NO: 7 - protein, 876 aa, Bacillus smithii
VKKKLILIDGNNIAYRAFFALPLLNNEKGIHTNAIYGFTMMLNKILEEEKPTHMLVAFDAGKTTFRHETFKEYKGGRQKTPPELSEQFPFIRDLLKSFHIPQFELENYEADDIIGTLSLEAEKKDFEIKIYSGDKDLTQLASEKTTVCLCRKGITDIEEYTPEHVKEKYGLTPQQIIDMKGLMGDSSDNIPGVPGIGEKTAIKLLKEFETVEKVVDSIDEISGKKLKERLQEHKQQALMSKELATIKRDAPLGITVDELEYQGPDWEKVQSIYKELGFQSLLEKIDQTQETEVQPLEKLEYQTVEEITEDLFEQENSFYLEMIGENYFISDIVGMAVQNEKGIFYLPTESTLKSPVFKKWAEDETKKKTVFDAKRTVIALRRHGIELKGIEFDLLLASYLINPSESPSDFADVAKLHGFNEVQSDEAVYGKGAKLKLPDREIYEEHIARKAVGLTKLAETCRDVLKENDQLSLFYDLEMPLALILADMEWTGVKVDVDRLTEMGDELHNRLQEIEKEIYELAGQEFNINSPKQLGHILFEKMGLPVIKKTKTGYSTSADVLEKLESSHEIVRYILEYRQLGKLQSTYIDGLLKVVHQNTHKVHTRFNQALTQTGRLSSADPNLQNIPIRLEEGRKIRQAFVPSEKDWVIFSADYSQIELRVLAHISGDQKLIEAFREDMDIHTKTAMDVFHVQKEEVTSNMRRQAKAVNFGIVYGISDYGLSQNLGITRKEAGQFIERYFASYPDVKEYMDEIVREAKRKGYVTTLLHRRRYLPEITSRNFNVRSFAERTAMNTPIQGSAADIIKKAMIDMAERLKKEQLKSRMLLQVHDELIFEVPPDEIETMKKIVPDVMEHAVELKVPLKVDYAYGPTWYDAK

SEQ ID NO: 8 - DNA, 1743 bp, Bacillus smithii
  CDS (1)..(1743)
atgaaattggagtatcaaactgtcgaagaaatcacggaggatttgttcgaacaggaaaattctttttatttagagatgattggtgaaaattatttcattagcgacattgtcggtatggctgttcaaaatgaaaaaggaattttttatttgccgacagaatcaactttaaaatctcctgtttttaaaaaatgggctgaagacgaaacgaaaaagaaaacggtgtttgatgctaagcgtacggttattgccttgcggaggcatggaattgaactgaaagggattgaatttgatttactgctagcttcttatttaattaacccgtctgaatccccttccgattttgccgatgtagctaagcttcatggttttaatgaagtgcaatccgacgaagcggtctacggaaaaggggcgaaattaaagctcccggacagagaaatttatgaggagcatattgcaagaaaagcggtggggcttactaaattggcggaaacatgtcgagatgttctgaaagaaaatgatcagctctctcttttttacgatttagaaatgccgcttgctttgattttggccgatatggaatggacaggcgtaaaggtggacgtggatcggttgacagaaatgggtgacgaacttcacaacaggctgcaggaaatcgaaaaggaaatctatgaattggccggacaagagtttaacattaactctcctaaacaactagggcatattttatttgaaaaaatggggctgccggtgataaagaaaacgaaaacaggttattccacatccgccgatgtattggaaaaattggaaagcagtcatgaaatcgttcgctacattttggagtaccgccagctaggaaaattgcaatccacttatatagatgggctgttgaaagtcgttcaccaaaatactcataaagtgcatactcgctttaatcaagctcttacacagacaggcagactgagttccgcagatcctaatttgcaaaacatcccgatcagactcgaagaaggaaggaaaatccgccaagcttttgtcccgtctgaaaaagattgggtcatcttttcagcggattattcacaaatcgaattgcgggttctcgctcatatatccggtgatcaaaagcttattgaagccttccgagaggatatggatatccacaccaaaacggccatggatgtttttcatgtacaaaaagaagaagtgacatccaatatgaggagacaggcgaaagccgttaattttgggattgtctatggaatcagcgattatggactctcacaaaatttagggattacgagaaaggaagccggtcagtttattgaacgttattttgcttcttatccggatgtaaaagaatatatggatgagattgttagagaagcgaaacgaaaaggttatgtaaccacattgcttcatagaagacgatatttgccggagattacaagccgaaatttcaatgtacgcagctttgcagagcggacggccatgaatacgccaatacaaggaagcgctgctgatattattaaaaaagcaatgattgatatggcagaacgattgaagaaagaacagcttaaatcgagaatgcttcttcaagtgcatgatgaattgatttttgaagttcctcccgatgagatagaaacgatgaaaaaaatcgtaccagatgtaatggaacatgcggttgaattgaaagttccgctaaaagtggattatgcctatggccccacttggtatgatgctaaatag

SEQ ID NO: 9 - protein, 580 aa, Bacillus smithii
MKLEYQTVEEITEDLFEQENSFYLEMIGENYFISDIVGMAVQNEKGIFYLPTESTLKSPVFKKWAEDETKKKTVFDAKRTVIALRRHGIELKGIEFDLLLASYLINPSESPSDFADVAKLHGFNEVQSDEAVYGKGAKLKLPDREIYEEHIARKAVGLTKLAETCRDVLKENDQLSLFYDLEMPLALILADMEWTGVKVDVDRLTEMGDELHNRLQEIEKEIYELAGQEFNINSPKQLGHILFEKMGLPVIKKTKTGYSTSADVLEKLESSHEIVRYILEYRQLGKLQSTYIDGLLKVVHQNTHKVHTRFNQALTQTGRLSSADPNLQNIPIRLEEGRKIRQAFVPSEKDWVIFSADYSQIELRVLAHISGDQKLIEAFREDMDIHTKTAMDVFHVQKEEVTSNMRRQAKAVNFGIVYGISDYGLSQNLGITRKEAGQFIERYFASYPDVKEYMDEIVREAKRKGYVTTLLHRRRYLPEITSRNFNVRSFAERTAMNTPIQGSAADIIKKAMIDMAERLKKEQLKSRMLLQVHDELIFEVPPDEIETMKKIVPDVMEHAVELKVPLKVDYAYGPTWYDAK

SEQ ID NO: 10 - DNA, 126 bp, Artificial Sequence
  Description of Artificial Sequence Synthetic
gcagcagaggggacatgaaatagttgtcctagcacctgacgcctcgttgtacatcagagacggagcattttacaccttgaagacgtaccctgtgccattccaaagggaggatgtgaaagagtcttt

SEQ ID NO: 11 - DNA, 30 bp, Artificial Sequence
  Description of Artificial Sequence Synthetic
acaacgaggcgcagcagaggggacatgaaa

SEQ ID NO: 12 - DNA, 30 bp, Artificial Sequence
  Description of Artificial Sequence Synthetic
ttgaagacgtaaagactctttcacatcctc

SEQ ID NO: 13 - DNA, 16 bp, Artificial Sequence
  Description of Artificial Sequence Synthetic
tgtgccattccaaagg